(19) United States
(12) Patent Application Publication
(10) Pub. No.: US 2024/0016436 A1
(43) Pub. Date: Jan. 18, 2024

(54) BRAIN INJURY REHABILITATION METHOD UTILIZING BRAIN ACTIVITY MONITORING

(71) Applicant: AXEM NEUROTECHNOLOGY INC., Halifax (CA)

(72) Inventors: Tony Joseph Gerald INGRAM, Dartmouth (CA); Christopher FRIESEN, Winnipeg (CA)

(21) Appl. No.: 18/040,423

(22) PCT Filed: Aug. 18, 2021
(86) PCT No.: PCT/CA2021/051141

§ 371 Date: Feb. 2, 2023

Related U.S. Application Data
(60) Provisional application No. 63/067,351, filed on Aug. 19, 2020.

(51) Int. Cl. A61B 5/378; A61B 5/00; A61B 5/384; A61B 5/375

(52) U.S. Cl. CPC A61B 5/378; A61B 5/0075; A61B 5/384; A61B 5/375

(57) ABSTRACT

The present invention provides a system and method of monitoring brain injury rehabilitation and a rehabilitation method which utilizes a non-invasive monitoring device.

### FIELD OF THE INVENTION

This disclosure relates generally to rehabilitation. In particular, brain injury rehabilitation methods which utilize brain activity monitoring.

### BACKGROUND

Individuals with an acquired brain injury (such as a stroke) often have mobility impairments, requiring intensive physical rehabilitation. Rehabilitation promotes recovery by leveraging neuroplasticity (i.e. the brain's ability to change). Prior art methods which promote or monitor treatments/rehabilitations are described in the following: US20090233769A1 describes a system to encourage the performance of remote rehabilitation exercises; U.S. Pat. No. 9,081,890B2 describes a rehabilitation training system and method provide rehabilitation-related information to the patient to provoke a rehabilitation intent of the patient; U.S. Pat. No. 9,311,789B1 describes use of motion-tracking to provide automated feedback during rehabilitation and US20180365385A1 describes an app-based method for evaluating patient's responses to medical treatments

Brain activity metrics may be used to predict recovery, track progress, and compare the effects of different exercises, potentially allowing clinicians to better tailor therapy to individual patients. See for example U.S. Pat. No. 8,380,314B2 which describes a system whereby brain activity is used to dictate what treatments are provided to a patient.

A number of methods of measuring brain activity are known in the art. Electroencephalography (EEG) which measures electrical activity. See for example U.S. Pat. No. 9,532,748 which teaches portable systems for brain activity recording, storage, analysis and neurofeedback. Near infrared spectroscopy which measures relative changes in oxygen concentration in the brain. Brain activity requires oxygen to use energy, which is known as the hemodynamic response and is the basis for many brain imaging technologies. When a user moves their left hand, the concentration of oxygen will increase in the right motor cortex in the area that controls the hand. The more muscle recruitment and the more complex the movement, the greater the oxygen change. See for example, WO2020006647A1 (incorporated herein by reference) which teaches a method and apparatus for monitoring brain activity of a user the apparatus includes a plurality of spatially separated emitters operable to generate infrared radiation.

There remains a need for methods of brain injury rehabilitation which utilize remote brain activity monitoring.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An objection of the present invention is to provide brain injury rehabilitation which utilizes brain activity monitoring. In one aspect of the present invention, there is provided a method of brain injury rehabilitation, the method comprising: detecting, by a non-invasive monitoring device, brain activity of a patient when the patient is performing the one or more rehabilitation exercises or activities, or at rest; sending collected brain activity to a server computing device in communication with the monitoring device; analyzing, by the server computing device, the collected brain activity data to identify various patterns of brain activation, and determining, based on the identified patterns of brain activation, any modifications to the rehabilitation exercises or activities and/or sending feedback based on the identified patterns of brain activation to the patient and/or the therapist overseeing the rehabilitation activity.

In certain embodiments, the method further comprising recording a video of the patient performing rehabilitation exercises and deriving kinematic information regarding the patient; wherein the modifications and/or the feedback is based on the patterns of brain activation and/or kinematic information.

In certain embodiments, the format of the feedback is dependent on party receiving the feedback and/or exercise being performed.

In certain embodiments, the feedback based on the identified patterns of brain activation is modified over time for a given exercise, based on the identified patterns of brain activation for that particular patient during that particular exercise.

In certain embodiments, the one or more rehabilitation exercises or activities is provided in a gaming experience and feedback to the patient is within the gaming experience.

In certain embodiments, the one or more rehabilitation exercises or activities is provided with multimedia content that has been provided by a friend/family member/caregiver of the patient.

In one aspect of the present invention, there is provided a method of monitoring brain injury rehabilitation, the method comprising: detecting, by a non-invasive monitoring device, brain activity of a patient during a rehabilitation activity, exercise, or at rest; sending collected brain activity to a server computing device in communication with the monitoring device; analyzing, by the server computing device, the collected brain activity data to identify various patterns of brain activation; comparing, by the server computing device, the pattern of brain activation to a control pattern of brain activation and/or a previously determined pattern of brain activation of the patient to determine any change in pattern of brain activation and/or determine effectiveness of the rehabilitation exercise or activity.

In certain embodiments, the method further comprising recording a video of the patient performing rehabilitation exercises and deriving kinematic information regarding the patient; wherein the determination of effectiveness of the rehabilitation exercise or activity is based on the pattern of brain activation and kinematic information.

In certain embodiments, the method further comprises modifying the rehabilitation exercise or activity if the rehabilitation exercise or activity was determined to be noneffective.

In certain embodiments, the non-invasive brain activity monitoring device is selected from the group consisting of an EEG-based brain activity monitoring device, near infrared spectroscopy (NIRS)-based device and MRI device.

### BRIEF DESCRIPTION OF THE DRAWINGS

These and other features of the invention will become more apparent in the following detailed description in which reference is made to the appended drawings.

FIG. 1 illustrates a system for use in brain injury rehabilitation which utilizes brain activity monitoring.

FIG. 2 illustrates an example of feedback from an embodiment of the method which utilizes a gaming experience. In this embodiment an image is revealed as more exercise is completed (and/or more brain activation occurs).

FIG. 3 provides a perspective view of an embodiment of a near infrared spectroscopy-based device for monitoring brain activity of the present invention.

FIGS. 4A-4Q illustrate various views of an embodiment of a near infrared spectroscopy-based device for monitoring brain activity of the present invention. A) Back of the Device view; B) Battery Compartment view; C) Battery Compartment Side view; D) Battery Compartment view; E) Cross section through front plane view; F) Cross section through right plane whole headset view; G) Cross Section Through Right Plane view; H) Electronics and Frame only view; I) Headset Back view; J) Headset Top view; K) Headset underside view; L) Back isometric view; M) front isometric view; N) front isometric view; O) front semi-isometric view; P) underside view and Q) underside view.

FIGS. 5A and 5B provide photographs of parts of an embodiment of a near infrared spectroscopy-based device for monitoring brain activity of the present invention.

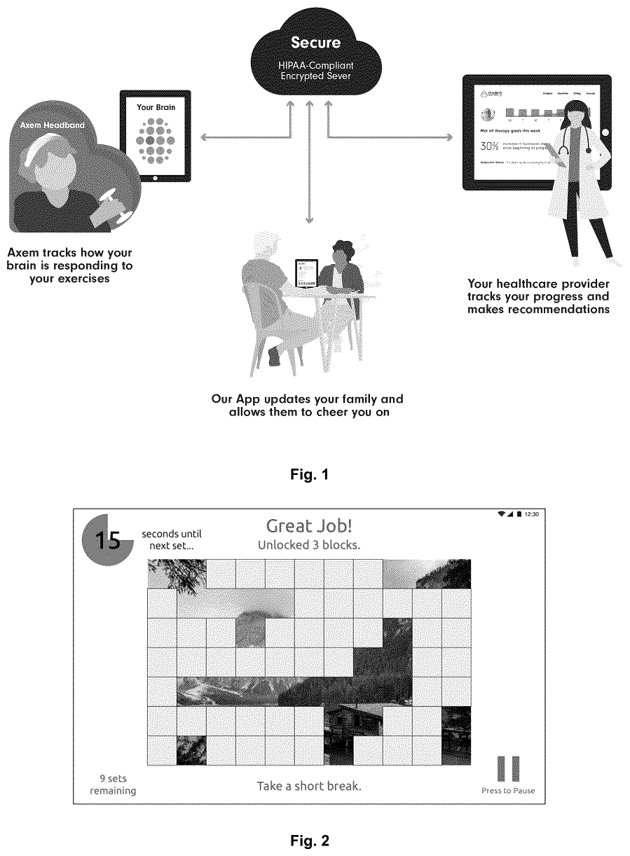

### DETAILED DESCRIPTION

The present invention provides systems and methods for brain injury rehabilitation which utilize brain activity monitoring alone or in combination with other biometric parameters such as kinematic information. As used herein, brain injury may include brain injury resulting from strokes including but not limited to ischemic and hemorrhagic strokes and/or traumatic brain injuries. In certain embodiments, the system of the present invention may facilitate and enhance brain injury rehabilitation/neurorehabilitation by providing a rehabilitative brain-computer-interface (rehab-BCI; sometimes referred to as “neurofeedback”) system designed to be usable by survivors of brain injury independently and at home, with software (e.g. an App) that guides patients through their home rehabilitation and recovery program. In certain embodiments, the system of the present invention allows healthcare professionals to monitor and manage the home program in clinic or remotely. In certain embodiments, the system of the present invention allows patients to invite peers, friends and family to offer support, encouragement, and accountability.

The methods and systems of the present invention may improve rehabilitation effectiveness, improve patient engagement, and lead to increased voluntary effort (which may increase brain activation in relevant brain areas), increase compliance and thus increase the dose of rehabilitation.

### Methods

The present invention provides methods of brain injury rehabilitation.

In certain embodiments, the method of brain injury rehabilitation comprises: detecting, by a non-invasive monitoring device, brain activity of a patient when the patient is performing one or more rehabilitation exercises or activities, or at rest; optionally recording a second biometric parameter such as video of the patient performing rehabilitation exercises; analyzing the collected brain activity data to identify various patterns of brain activation and; optionally analyzing the second biometric parameter, such as analyzing the video of the patient's movements to derive kinematic information reflecting the patient's ability to complete the rehabilitation exercises; and determining based on identified patterns of brain activity and optionally the second biometric parameter any modifications to the rehabilitation exercises or activities and providing feedback.

Detecting, by a non-invasive monitoring device, brain activity of a patient when the patient is performing one or more rehabilitation exercises or activities

Brain activity may be measured using a variety of monitoring devices including but not limited to EEG-based brain activity monitoring devices, near infrared spectroscopy-(NIRS)-based devices and MRI devices. In certain embodiments, the monitoring device is a NIRS-based device. In other embodiments, the monitoring device is an EEG-based monitoring device.

The one or more rehabilitation exercises or activities may be a default exercises/activities or exercise program; automatically generated based on patient history based on a pre-defined set of rules; or exercises or activities inputted into the system by the patient's healthcare professional. Accordingly, in certain embodiments, the method further comprises providing one or more exercises. In certain embodiments, generating an rehabilitation exercise program comprising one or more exercises based on patient history based on a pre-defined set of rules

A particular exercise/activity or program of exercises/activities is selected to start and the system prompts the patient to start the exercise/activity. The prompt may be visual, auditory and/or tactile. Optionally, the system provides detailed instructions and/or video demonstrations on how to perform the exercise and/or activity.

Optionally, one or more other biometrics are monitored in addition to brain activity. Such biometrics include but are not limited to heart rate and body movements. In certain embodiments, a patient's movements are monitored. For example, the system of the present invention may utilize a camera on the device the patient is using (e.g. personal computer, tablet or smartphone) to record the patients' movement during rehabilitation exercises. Accordingly, in certain embodiments, the method comprises detecting, by a non-invasive monitoring device, brain activity of a patient when the patient is performing one or more rehabilitation exercises or activities, or at rest; and detecting a second biometric parameter.

In certain embodiments, the system of the present invention also records video of the patient performing rehabilitation exercises, and derives from this (using computer vision methods including but not limited to: Convolutional Neural Networks, Optical Flow Tracking, and Histogram Matching) kinematic information reflecting the patients ability to complete the rehabilitation exercises (and thus reflective of their current motor abilities, as it relates to the movements required to complete that rehabilitation exercise). Accordingly, in certain embodiments, the method comprises detecting, by a non-invasive monitoring device, brain activity of a patient when the patient is performing one or more rehabilitation exercises or activities, or at rest; and recording a video of the patient performing rehabilitation exercises.
b) analyzing the collected brain activity data to identify various patterns of brain activation and;

In certain embodiments, the patient's own neural pattern (and/or algorithms derived from the analysis of other patient's neural data) is used to derive an optimal feedback metric for that patient. In specific embodiments, motion capture (potentially enabled through computer vision based on the camera in a tablet being used for accessing an app associated with the invention) to inform and/or augment this feedback signal.

Non-limiting examples of patterns of brain activity include but are not limited to:
The ratio of brain activation between the two sides (or “hemispheres”) of the brain, known as “laterality”, during certain tasks and in certain areas of the brain can predict certain types of recovery—in the case of stroke, both absolute and relative (i.e., changes over time with a given patient) laterality of the primary motor cortex (M1) activation during a motor task is predictive of recovery of upper extremity motor function.
The correlation of activation at rest, known as “resting state functional connectivity” (rs-FC), at M1 both absolute and relative—i.e., changes over time with a given patient) is also predictive of upper extremity motor recovery.
Changes in the topology brain activation within cortical motor regions.
Changes over time in the degree of and/or consistency of brain activation in the contra-lesional motor cortex, when performing the same exercises across days/weeks/months.
Differences in the levels of hemodynamic activity in the contra-lesional motor cortex between the different exercises performed by a stroke survivor (with higher levels of contra-lesional activity indicative of more efficacious rehabilitation).
Changes in effective connectivity (i.e., the causal influence of one brain area on the level of activity in another brain area) between interhemispheric and/or primary and secondary cortical motor regions.

In embodiments that utilize functional MRI to monitor brain activity, both BOLD rsFC and laterality may be derived through this functional neuroimaging modality. Moreover, if fMRI is used additional information about intracortical connectivity within the sensorimotor system and/or cerebellum optionally is used as an additional biomarker or in combination with the aforementioned biomarkers.

In embodiments that utilize EEG in lieu of fNIRS; desynchronization in the alpha and/or beta bands (˜8-15 Hz) at the sensorimotor cortex is used in lieu of an increase in BOLD/relative oxyhemoglobin at the sensorimotor cortex.

In certain embodiments, video of the patient's movements taken while performing the rehabilitation exercises/activities is also analyzed to derive kinematic information reflecting the patient's ability to complete the rehabilitation exercises (and thus reflective of their current motor abilities, as it relates to the movements required to complete that rehabilitation exercise). In specific embodiments, computer vision methods selected from the group consisting of Convolutional Neural Networks, Optical Flow Tracking, and Histogram Matching are used to derive kinematic information. Accordingly, in certain embodiments, the method further comprises recording video of the patient performing the rehabilitation exercises and deriving kinematic information.
c) determining any modifications to the rehabilitation exercises or activities and/or providing feedback to the patient; optionally providing feedback/report to a third party.

In certain embodiments, the rehabilitation program is modified in response to monitored brain activity and optionally other biometric information including but not limited to kinematic information. The rehabilitation program may be modified in one or more aspects including but not limited to changing exercises or rehabilitation tasks (e.g. reaching forward), changing the technique (alterations of movement patterns to achieve the same movement goal—e.g. reaching forward with more shoulder external rotation or “with elbow turned out”) and/or changing the parameters of exercises (e.g. timing, reps and sets, frequency). In certain embodiments, the modification(s) to the rehabilitation program is selected to increase brain activity and/or shifts in biomarkers that are deemed desirable by the clinician.

In certain embodiments, the rehabilitation program is modified (in the manner specified above) in response to kinematic information (derived from video of the patient captured during the performance of rehabilitation exercises).

In certain embodiments, the rehabilitation program is modified (in the manner specified above) in response to a combination of monitored brain activity and kinematic information.

In certain embodiments, the rehabilitation program is automatically modified based on the on brain activity and/or kinematic information based on a pre-defined set of rules.

In certain embodiments, the brain activity data and/or kinematic information is automatically sent to the patient's healthcare provider. In such embodiments, the patient's healthcare provide may input modifications to the rehabilitation program or exercises. In certain embodiments, the system provides recommended modifications based on the brain activity and/or kinematic information based on a pre-defined set of rules

Feedback may improve patient engagement, and lead to increased voluntary effort (which may increase brain activation in relevant brain areas), increase compliance and thus increase the dose of rehabilitation. Accordingly, in certain embodiments, the system provides feedback to the user. The feedback may be provided before, during or after the rehabilitation exercises. The feedback may be based on brain activity, kinematic data or both brain activity and kinematic data.

Feedback may be in the form of visual, auditory, and/or tactile feedback. In certain embodiments, the feedback is provided in real-time or after a short delay. In certain embodiments, the patient and/or therapist chooses how the feedback is presented. In certain embodiments, the format and/or content of the feedback differs depending on the party receiving the feedback. For example, a therapist may receive a report detailing brain activity patterns while a patient may receive feedback in the form of positive encouragement and/or information related to exercises and/or rehabilitation tasks to perform. In certain embodiments, the feedback is specific to the exercise or activity being performed or the body part the exercise or activity targets.

In certain embodiments, the systems and methods of the present invention utilizes a gaming experience. Accordingly, in certain embodiments, the patient feedback is within the gaming experience. In specific embodiments, the patient feedback is part of game mechanics. In specific embodiments, the patient feedback is presented in reference to an explicit win state the patient is encouraged to achieve. This may encourage the patient to “improve” their brain activity patterns in this particular way.

Research suggests that drivers of rehabilitation compliance include support and monitoring, a sense of accountability, and social engagement. Accordingly, in certain embodiments of the present invention, the system and method allows patients to invite peers/friends/family to observe and support their progress. In certain embodiments, the system and methods allow peers/friends/family to observe exercise adherence (e.g. how many logins, how long a patient was logged in, how many minutes of exercise was completed, and so on) and comment and congratulate the patient on their efforts. In certain embodiments, the system and methods allow peers/friends/family to provide motivating content to incentivize adherence. For example, family photos may be uploaded that can be revealed with good compliance and/or utilized in rehab game mechanics. Accordingly, in certain embodiments, the one or more rehabilitation exercises or activities are presented in combination multimedia content (e.g., photographs, short notes) that has been provided (via a web or mobile app interface allowing them to upload multimedia content) by a friend/family member/caregiver of the patient.

Some patterns of brain activity are known to be biomarkers of brain injury (including but not limited to stroke) recovery. Accordingly, the present invention provides a method of monitoring rehabilitation/recovery and/or predict prognosis. Accordingly, in certain embodiments, the method of monitoring brain injury rehabilitation, the method comprising: detecting, by a non-invasive monitoring device, brain activity of a patient during a rehabilitation activity, exercise, or at rest; sending collected brain activity to a server computing device in communication with the monitoring device; analyzing, by the server computing device, the collected brain activity data to identify various patterns of brain activation; comparing, by the server computing device, the pattern of brain activation to a control pattern of brain activation and/or a previously determined pattern of brain activation of the patient to determine any change in pattern of brain activation and/or determine effectiveness of the rehabilitation exercise or activity. This information may result in changes to the rehabilitation and recovery program and/or be used to indicate recovery to other stakeholders (e.g. insurance companies).

The collected data may be of interest to researchers and/or clinicians or may be used to identify biomarkers of brain injury and/or brain injury recovery. Accordingly, in certain embodiments, there is provided a method of generating a database of brain activity data and a database of brain activity data. In certain embodiments, the data is anonymized. In specific embodiments, collected data is stored in a secure (e.g. HIPAA compliant) cloud storage which may be accessed remotely by researchers and/or healthcare professionals.

### System

The present invention further provides a system for use in brain injury rehabilitation. The system comprises one or more brain activity monitoring devices in communication with a server computing device. Optionally, the system further comprises or is in communication with one or more means to record patient movement such as digital camera or smartphone, tablet or computer. In certain embodiments, a patient's user device is used to record patient movement.

In certain embodiments, the system of the present invention is in communication with one or more user devices, including but not limited to patient user devices, healthcare professional user devices and/or user devices of other third-parties, such as family or friends of the patient providing feedback. The user devices may include but are not limited to tablets, smartphones, smartwatches and personal computers.

Optionally, the system further comprises one or more databases. The one or more databases may store patient data including for example patient history and rehabilitation plan, brain activity data and brain activity patterns and/or rehabilitation exercises, activities and/or program; brain activity data and/or brain activity patterns of normal individuals and those with brain injury; standard rehabilitation exercises and/or programs.

Accordingly, the system of the present invention allows for one or more portable brain activity monitoring devices and optionally other devices, such as cameras to collect data. The data is sent to a server computing device, optionally a cloud-based server computing device for analysis and the analysed data and/or feedback plan based on the analysed data is forwarded to one or more user devices (including but not limited to the patient's device and third-party devices such as the healthcare provider's device and devices of family).

The brain activity monitoring device may be any non-invasive brain activity monitoring device including but not limited to EEG-based brain activity monitoring devices, near infrared spectroscopy-(NIRS)-based devices and MRI devices. Near infrared spectroscopy-based devices, EEG and MRI devices are known in the art. For example, WO2020006647A1 (incorporated herein by reference) teaches a method and apparatus for monitoring brain activity of a user the apparatus includes a plurality of spatially separated emitters operable to generate infrared radiation.

Accordingly, in certain embodiments, the monitoring device is a NIRS-based device. In specific embodiments, the NIRS-based device is portable device. In more specific embodiments, the NIRS-based device is designed for home use. In more specific embodiments, the device is designed to be useable by survivors of brain injury independently. For example, the device may be configured to allow use by individuals having motor, including fine motor, or cognitive impairments. In certain embodiments, the device is configured as a headband that can be easily put on and off the head and that does not require specific positioning of sensors. In certain embodiments, the monitoring device is App controlled. Any appropriate device including but not limited to a tablet, smartphone or smartwatch may be used to run the App. In certain embodiments, the App may be configured to be useable by survivors of brain injury independently.

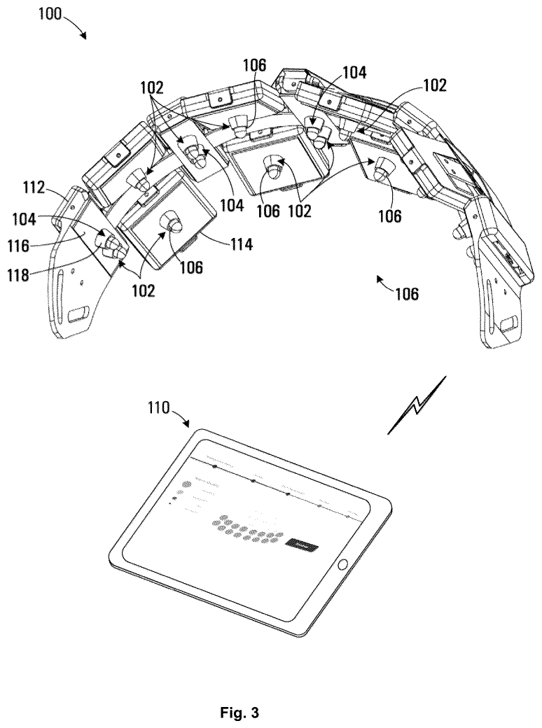

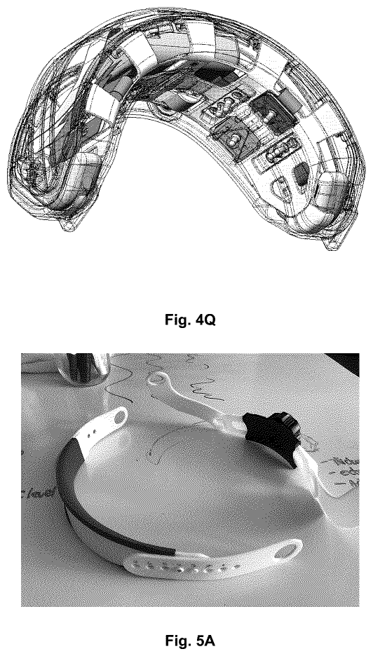

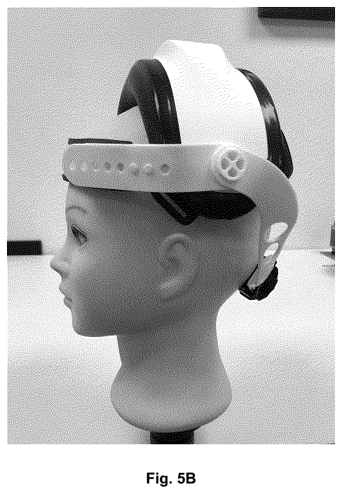

Non-limiting examples of NIRS-based monitoring devices are illustrated in FIGS. 3 to 5B.

Referring to FIG. 3, the NIRS-based monitoring device 100 of this embodiment includes a plurality of spatially separated near infrared radiation emitters 102 and a plurality of spatially separated near infrared radiation detectors 104. Each one of the emitters 102 and the detectors 104 have an associated light pipe 106, which is operable to couple near infrared radiation from the emitter into the user's scalp or to couple near infrared radiation from the scalp to the detector. In this embodiment the emitters 102 are mounted within a headset 108 operable to support the plurality of emitters 102 and plurality of detectors 104 in contact with the user's scalp when the headset is worn by the user such that each of the light pipes 106 contact the user's scalp. Each detector 104 is operable to produce a signal representing an intensity of near infrared radiation generated by a selectively actuated one of the plurality of emitters 102 and received at the detector after traveling on a path through underlying brain tissue. The near infrared radiation from each emitter 102 penetrates the scalp and skull and travels along a path through respective portions of underlying brain tissue, which reflects the radiation back to one or more of the detectors 104.

In certain embodiments, the headset 108 is controlled via an App on a tablet 110.

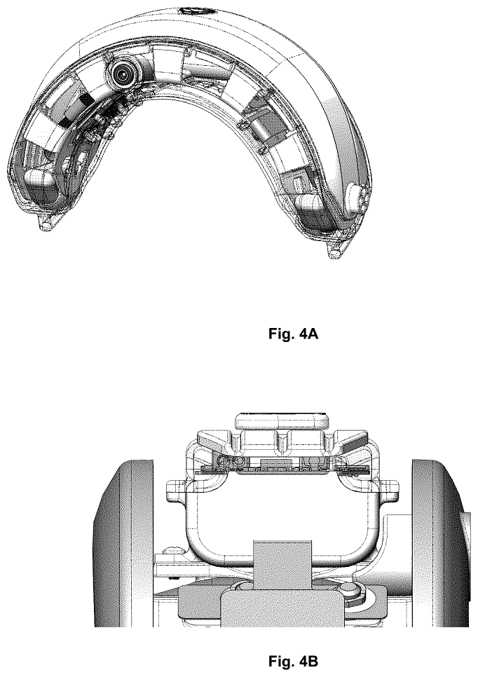

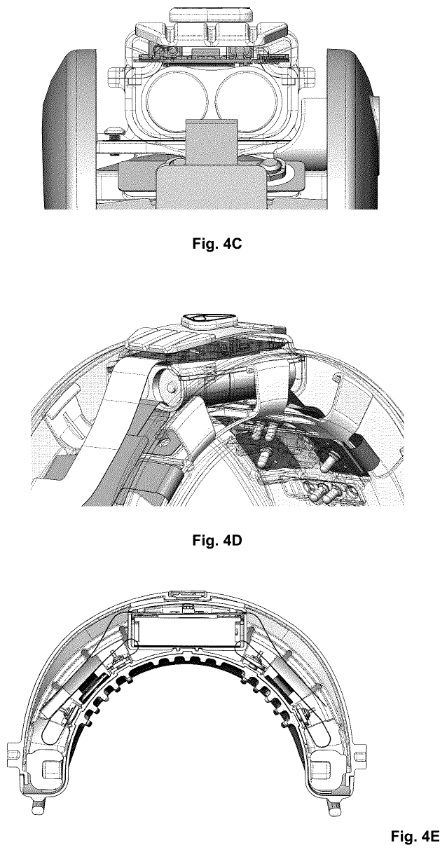

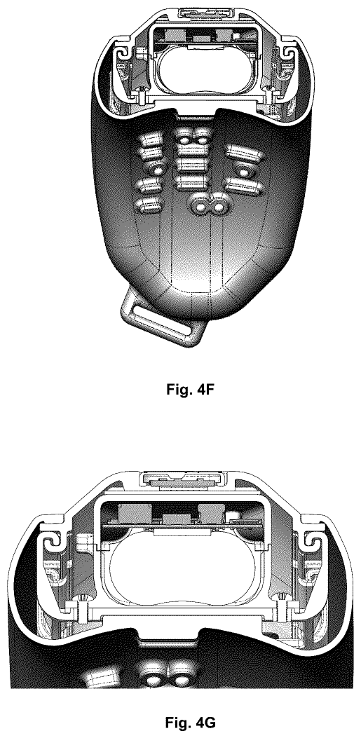

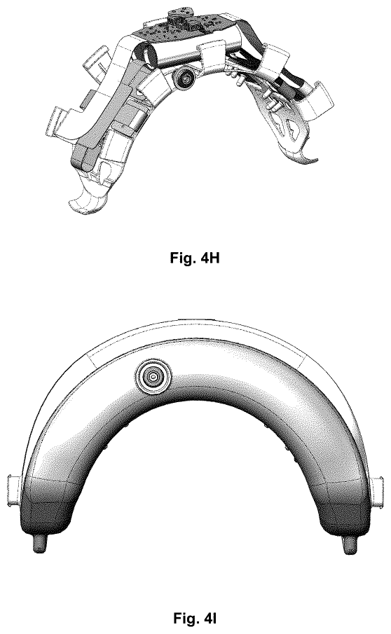

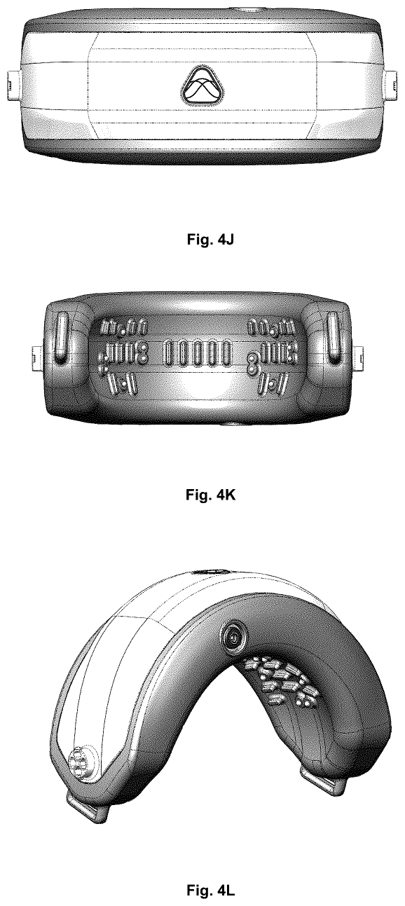

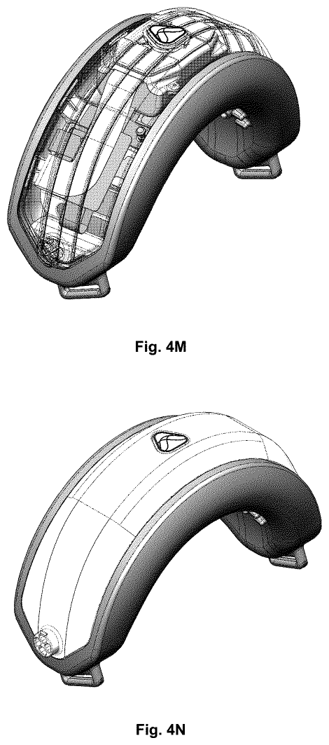

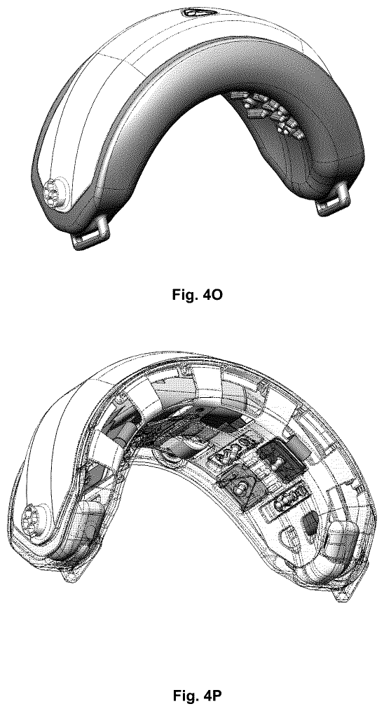

An alternative embodiment of a NIRS-based device is illustrated in FIGS. 4A-Q, 5A and 5B. In this embodiment, the design is more enclosed than device of FIG. 3. In specific embodiments, the device is enclosed at least partially in a semi-transparent or transparent covering. This allows for it to be more easily handled and wiped down for cleaning.

A worker skilled in the art would readily appreciate that the position of the sensors will dictate the portion of the brain activity is being measured. Moreover, such a worker would further appreciate that different parts of the brain control different areas of the body. Accordingly, in certain embodiments, the sensors are configured to only measure brain activity associated with certain parts of the body. In specific embodiments, the device only has four measurement locations on each lateral side. Accordingly, the device targets only the upper extremities and does not measure lower extremities.

To gain a better understanding of the invention described herein, the following examples are set forth. It will be understood that these examples are intended to describe illustrative embodiments of the invention and are not intended to limit the scope of the invention in any way.

### EXAMPLE

### Example 1 of System Usage

1. Patient is assigned default exercise program, using the default algorithm for the provision of brain activity feedback.
2. Patient performs the default exercise program while wearing the brain monitoring device and optionally records video of performing the exercises.
3. Based on identified patterns of brain activity (measured via brain measurement device during exercises) and optionally kinematic information (derived from video taken using the tablet's camera during exercises), the exercises presented to the patient are changed such that they are presented more challenging exercises.
4. The patient's physician and/or therapist are notified of this change.

### Example 2 of System Usage

1. Patient is assigned default exercise program, using the default algorithm for the provision of brain activity feedback.
2. Patient performs the default exercise program while wearing the brain monitoring device and optionally records video of performing the exercises.
3. Based on identified patterns of brain activity (measured via brain measurement device during exercises) and kinematic information (derived from video taken using the tablet's camera during exercises) suggesting a high recovery potential, the brain activity feedback presented to the patient (for all exercises) changes such that up-regulation of contra-lesional motor cortex brain activity up-regulation becomes disincentivized (due to the fact that this brain activity is known to be only beneficial when patient's have low recovery potential and lack sufficient corticospinal tract integrity (Di Pino et al. (2014). Nature Reviews Neurology, 10(10), 597-608).
4. The patient's physician and/or therapist are notified of this change.

### Example 3 of System Usage

1. Patient is assigned default exercise program, using the default algorithm for the provision of brain activity feedback.
2. Patient performs the default exercise program while wearing the brain monitoring device and optionally records video of performing the exercises.
3. Based on identified patterns of brain activity (measured via brain measurement device during exercises) and kinematic information (derived from video taken using the tablet's camera during exercises) the brain activity feedback signal presented during exercises only involving the fingers is altered to include a smaller subset of measurement locations, due to the lower proportion of the motor cortex this task engages.
4. The patient's physician and/or therapist are notified of this (above #5) change.

### Example 4 of System Usage

1. Patient is assigned default exercise program, using the default algorithm for the provision of brain activity feedback.
2. Patient performs the default exercise program while wearing the brain monitoring device and optionally records video of performing the exercises.
3. Based on identified patterns of brain activity (measured via brain measurement device during exercises) and kinematic information (derived from video taken using the tablet's camera during exercises), the requested volume of rehabilitation exercises requested of the patient is increased.
4. The patient's physician and/or therapist are notified of this (above #7) change.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A method of brain injury rehabilitation, said method comprising:
detecting, by a non-invasive monitoring device, brain activity of a patient when said patient is performing said one or more rehabilitation exercises or activities, or at rest;
sending collected brain activity to a server computing device in communication with said monitoring device;
analyzing, by said server computing device, said collected brain activity data to identify various patterns of brain activation, and
determining, based on said identified patterns of brain activation, any modifications to said rehabilitation exercises or activities and/or sending feedback based on the identified patterns of brain activation to said patient and/or the therapist overseeing said rehabilitation activity.

2. The method of claim 1, further comprising recording a video of the patient performing rehabilitation exercises and deriving kinematic information regarding said patient; wherein said modifications and/or said feedback is based on said patterns of brain activation and/or kinematic information.

3. The method of claim 1, wherein format of said feedback is dependent on party receiving the feedback and/or exercise being performed.

4. The method of claim 1, wherein said feedback based on the identified patterns of brain activation is modified over time for a given exercise, based on the identified patterns of brain activation for that particular patient during that particular exercise.

5. The method of claim 1, wherein said providing one or more rehabilitation exercises or activities is in a gaming experience and feedback to said patient is within said gaming experience.

6. The method of claim 1, wherein said providing one or more rehabilitation exercises or activities includes multimedia content that has been provided by a friend/family member/caregiver of the patient.

7. A method of monitoring brain injury rehabilitation, said method comprising:
detecting, by a non-invasive monitoring device, brain activity of a patient during a rehabilitation activity, exercise, or at rest;
sending collected brain activity to a server computing device in communication with said monitoring device;
analyzing, by said server computing device, said collected brain activity data to identify various patterns of brain activation;
comparing, by said server computing device, said pattern of brain activation to a control pattern of brain activation and/or a previously determined pattern of brain activation of said patient to determine any change in pattern of brain activation and/or determine effectiveness of the rehabilitation exercise or activity.

8. The method of claim 7, further comprising recording a video of the patient performing rehabilitation exercises and deriving kinematic information regarding said patient; wherein said determination of effectiveness of said rehabilitation exercise or activity is based on said pattern of brain activation and kinematic information.

9. The method of claim 7, further comprising modifying said rehabilitation exercise or activity if said rehabilitation exercise or activity was determined to be suboptimal.

10. The method of claim 1, wherein said non-invasive brain activity monitoring device is selected from the group consisting of an EEG-based brain activity monitoring device, near infrared spectroscopy (NIRS)-based device, MRI device and a combination of EEG and NIRS based device.
